(19) United States
(12) Patent Application Publication
(10) Pub. No.: US 2023/0190937 A1
(43) Pub. Date: Jun. 22, 2023

(54) IRON COMPLEXES OF ESTERIFIED DERIVATIVES OF NATURAL PRODUCTS, AND METHODS OF USE

(71) Applicant: CORNELL UNIVERSITY, Ithaca, NY (US)

(72) Inventors: Amin Zarei, Ithaca, NY (US); Leila Khazdooz, Ithaca, NY (US); Alireza Abbaspourrad, Ithaca, NY (US)

(21) Appl. No.: 17/981,922

(22) Filed: Nov. 7, 2022

Related U.S. Application Data
(60) Provisional application No. 63/276,234, filed on Nov. 5, 2021.

(51) Int. Cl. A61K 47/44; A61K 33/26; A61K 47/14; A61P 3/02; A23L 33/115; A23L 33/16; A23L 33/00; A23D 9/007

(52) U.S. Cl. CPC A61K 47/44; A23D 9/007; A23L 33/16; A23L 33/40; A23L 33/115; A61K 33/26; A61K 47/14; A61P 3/02

(57) ABSTRACT

Consumable (edible) iron-containing compositions containing precisely or at least the following components: (i) an edible oil; (ii) ionic iron, typically as an iron salt or iron complex; and (iii) an esterified derivative of a natural product, such as an esterified version of curcumin, catechin, or ascorbic acid. Methods for preparing these compositions are also disclosed. Food products containing these compositions are also disclosed. Also disclosed herein are methods for treating a subject having an iron deficiency by administering the iron-containing composition to the subject.

### CROSS REFERENCE TO RELATED APPLICATIONS

This application claims the benefit of priority from U.S. Provisional Application No. 63/276,234, filed on Nov. 5, 2021, which is herein incorporated by reference in its entirety.

### FIELD OF THE INVENTION

The present invention generally relates to iron-containing compositions that can be incorporated into supplements and foods for human or animal consumption wherein the iron is in bioavailable form. The present invention more particularly relates to compositions containing a complex of an iron salt and a natural product, wherein the complex is dissolved or dispersed in an edible oil.

### BACKGROUND OF THE INVENTION

Iron is an essential micronutrient that is involved in a number of vital biological processes, especially in oxygen transport, maintaining and development of the normal function of neurons, DNA synthesis, and cellular respiration to produce energy. As a worldwide public health issue, nowadays, around 30% of the world population has iron deficiency (H. T. Lorinczova, et al., Nutrients, 13:2300, 2021). This deficiency harms growth and metabolism in children and can lead to impairment in their cognitive and physical development. Moreover, iron deficiency can endanger physical and cognitive performance in adults, accompanied by fatigue, reduced mood, and impaired quality of life (J. D. Haas et al., The Journal of Nutrition 2001;131:676S-690S). Insufficient nutrient intake and the negative effect of dietary iron inhibitors like polyphenols are prime contributors to the decrease in iron absorption.

There currently remains no viable technology for iron fortification in the food industry. The current practice is to add iron salt in a high dosage to food products. However, particularly in the case of foods that require cooking, the added iron becomes oxidized and precipitates, and is thus no longer bioavailable. Thus, there would be a significant benefit in consumable iron compositions that are highly bioavailable as well as thermally stable.

### SUMMARY OF THE INVENTION

In a first aspect, the present disclosure is directed to consumable iron-containing compositions containing precisely or at least the following components: (i) an edible oil; (ii) ionic iron, typically as an iron salt or iron complex; and (iii) an esterified derivative of a natural product. The edible oil may be any of the food grade oils known in the art, such as coconut oil, corn oil, canola oil, palm oil, grape seed oil, soybean, peanut oil, or olive oil, or a mixture thereof. The ionic iron may be in the ferrous (Fe⁺²) or ferric (Fe⁺³) form and is associated with or complexed with the esterified derivative of a natural product. The ionic iron may also be associated with a counteranion (e.g., halide, sulfate, and/or carboxylate). The natural product (i.e., before being esterified) is typically one that contains two or more hydroxy (OH) groups on one or more aromatic or aliphatic rings. After being esterified (i.e., with —C(O)R groups), the esterified natural product may contain a portion or all of its hydroxy groups esterified. The esterified natural product may be, for example, an esterified form of curcumin, catechin, or ascorbic acid.

In another aspect, the present disclosure is directed to methods of preparing the iron-containing compositions described above. In the methods, at least the esterified form of natural product, the edible oil, and ionic iron are combined, either in one step or in separate steps. A first method may include the following steps: combining the esterified form of natural product (e.g., an esterified form of curcumin, catechin, and/or ascorbic acid) and edible oil to form a mixture, and further combining the mixture with the ionic iron (or adding the ionic iron to the mixture) to form the iron-containing composition. A second method may include the following steps: (i) combining the esterified form of natural product (e.g., an esterified form of curcumin, catechin, and/or ascorbic acid) and edible oil to form a first mixture; dispersing the ionic iron in a co-solvent (e.g., ethanol or other food grade co-solvent) to form a second mixture; and combining the first and second mixtures (which may include adding the second mixture into the first mixture, or vice-versa) to form the iron-containing composition.

In another aspect, the present disclosure is directed to a food product containing any of the iron-containing compositions described above, wherein the composition may be produced by any of the methods described above. The food product may be, for example, a grain, grain-containing product, grain-derived product, vegetable, vegetable-containing product, vegetable-derived product, meat, meat-containing product, or meat-derived product.

In yet another aspect, the present disclosure is directed to a method of treating an iron deficiency in a subject by administering a therapeutic amount of any one of the iron-containing compositions described above to the subject. In certain embodiments, the subject may have a lower than optimal or deficient blood iron level, red blood cell count (e.g., hematocrit), or hemoglobin. In some embodiments, the subject has anemia.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. ATR FT-IR spectrum of curcumin dibutanoate.

FIG. 2. ¹H NMR (500 MHz) of curcumin dibutanoate in CDCl₃.

FIG. 3. Expanded ¹H NMR (500 MHz) of curcumin dibutanoate.

FIG. 4. ¹³C NMR (125 MHz) of curcumin dibutanoate in CDCl₃.

FIG. 5. ATR FT-IR of curcumin dioctanoate.

FIG. 6. ¹H NMR (500 MHz) of curcumin dioctanoate in CDCl₃.

FIG. 7. Expanded ¹H NMR (500 MHz) of curcumin dioctanoate.

FIG. 8. ¹³C NMR (125 MHz) of curcumin dioctanoate in CDCl₃.

FIG. 9. Solubility of curcumin, curcumin dibutanoate, and curcumin dioctanoate in coconut oil.

FIG. 10. UV-Vis absorption spectra of curcumin, curcumin dibutanoate, and curcumin dioctanoate in coconut oil.

FIG. 11. Soluble Fe-curcumin dioctanoate complex and Fe-curcumin dibutanoate complex in coconut oil.

FIG. 12. Formation of Fe-curcumin dibutanoate and Fe-curcumin dioctanoate in coconut oil by using ethanol as a cosolvent.

FIG. 13. a) Formation of Fe-curcumin dibutanoate in canola oil by using ethanol as a cosolvent. b) Comparison of Fe-curcumin dibutanoate in canola and coconut oils.

FIG. 14. Synthesis of Catechin-penta butanoate.

FIG. 15. FTIR of Catechin-penta butanoate.

FIG. 16. ¹HNMR of Catechin-penta butanoate in CDCI₃.

FIG. 17. ¹HNMR of Catechin-penta butanoate in CDCI₃.

FIG. 18. ¹³CNMR of Catechin-penta butanoate in CDCI₃.

FIG. 19. LC-MS of Catechin-penta butanoate.

FIG. 20. UV-Vis absorption spectra of Catechin & Catechin penta butanoate.

FIG. 21. Synthesis of Ascorbyl-tetra butanoate.

FIG. 22. FTIR of Ascorbyl-tetra butanoate.

FIG. 23. ¹HNMR of Ascorbyl-tetra butanoate in CDCI₃.

FIG. 24. ¹HNMR of Ascorbyl-tetra butanoate in CDCI₃.

FIG. 25. ¹³CNMR of Ascorbyl-tetra butanoate in CDCI₃.

FIG. 26. UV-Vis absorption spectra of ascorbyl-tetra butanoate in hexane.

### DETAILED DESCRIPTION OF THE INVENTION

In a first aspect, the present disclosure is directed to consumable (edible or food-safe) iron-containing compositions containing precisely or at least the following components: (i) an edible oil; (ii) ionic iron; and (iii) an esterified derivative of a natural product. In some embodiments, the composition includes only the foregoing three components. In other embodiments, the composition includes one or more other components, such as a non-toxic co-solvent, non-toxic surfactant, flavoring agent, coloring agent, anti-oxidant, and/or one or more other vitamins or nutrients.

The edible (food grade) oil can be any of the food grade oils known in the art. The food grade oil may be for human or animal consumption. The food grade oil may contain, for example, a mono-, di-, or tri-glyceride, or combination thereof, and may be, for example, a plant or animal derived oil. Some examples of plant oils include coconut oil, corn oil, canola oil, palm oil, grape seed oil, soybean, peanut oil, olive oil (e.g., extra virgin olive oil), almond oil, avocado oil, cottonseed oil, flax seed oil, sesame seed oil, walnut oil, safflower oil, sunflower oil, palm kernel oil, hemp seed oil, grape seed oil, rapeseed oil, lemon oil, cocoa butter, and orange oil, any of which may be refined or unrefined. The food grade oil may also be an artificial food grade oil, such as mineral oil or a fatty acid-substituted sugar (e.g., olestra). The food grade oil may also be a fish, krill, or algal oil, which are typically high in omega-3 fatty acids. In some embodiments, any one or more of the foregoing types of oils may be excluded from the composition. In some embodiments, the oil has a low moisture content, preferably no more than or less than 0.3, 0.2, 0.1, 0.05, 0.02, or 0.01% of water.

The ionic iron is typically an iron salt or iron complex or a combination of both. The ionic iron may be in the ferrous (Fe⁺²) or ferric (Fe⁺³) state. In some embodiments, the ionic iron in the composition does not form a metal-ligand complex with the esterified form of the natural product, although the ionic iron may be single-bonded to or otherwise associated with the esterified natural product without forming a metal-ligand complex. The term “metal-ligand complex,” as used herein, refers to a metal atom coordinated in bidentate or tridentate fashion to a ligand containing two or three coordinating groups, respectively. In other embodiments, the esterified form of the natural product complexes to the iron in bidentate or tridentate fashion. In some embodiments, the ionic iron is selected from FeCl₂•4H₂O, FeSO₄ (ferrous sulfate or iron(II) sulfate), FeCl₂ (ferrous chloride or iron(II) chloride), Fe(NO₃)₃ (ferric nitrate) or iron(III) nitrate), Fe(SO₄)₃ (ferric sulfate or iron(III) sulfate), FeC1₃ (ferric chloride or iron(III) chloride).

The esterified natural product is typically uncharged (neutral), but may, in some cases, be anionic, in which case the esterified natural product may serve as an anionic species which may partially or fully counterbalance the positive charge of the ionic iron. In the case of a neutral esterified natural product, the ionic iron is typically associated with one or more non-toxic anions (e.g., halide, sulfate, a carboxylate, or an anionic ligand different from the esterified natural product) to maintain charge neutrality. The halide is typically chloride or bromide. The carboxylate may be, for example, acetate, propionate, butyrate, or gluconate. Other types of non-toxic anions include picolinate and pyruvate.

The natural product (i.e., before being esterified) is typically one that contains two or more hydroxy (OH) groups on one or more aromatic or aliphatic rings. After being esterified, the esterified natural product may contain a portion or all of its hydroxy groups esterified. In some embodiments, the esterified natural product contains at least two groups capable of forming complexing coordination bonds with the iron. The at least two groups may be selected from, for example, carbonyl, hydroxy, thiol, sulfide, amino, and amide groups. In some embodiments, the esterified natural product may contain an enol moiety for coordinating to the iron atom. The esterified natural product typically has a molecular weight of at least or greater than 100, 150, 200, 250, or 300 g/mol or within a range therein. The esterified natural product may, in some embodiments, have a molecular weight up to or less than 300, 400, 500, 600, 700, 800, 900, or 1000 g/mol. The esterified natural product may be, for example, an esterified form of curcumin, catechin, or ascorbic acid. The term “esterified,” as used herein, generally refers to a hydroxy (OH) group on the natural product that has undergone esterification to result in an ester group (e.g., —OC(O)R) group, where R may be a hydrocarbon group, more typically a linear or branched alkyl or alkenyl group, containing 1, 2, 3, 4, 5, 6, 7, 8, 9, 10, 11, 12, 13, 14, 15, 16, 17, 18, 19, or 20 carbon atoms, or a number of carbon atoms within a range bounded by any two of the foregoing values (e.g., 1-20, 2-20, 3-20, 4-20, 1-12, 2-12, 3-12, 4-12, 1-6, 2-6, 3-6, or 4-6 carbon atoms).

In a first set of embodiments, the esterified natural product is an esterified form of curcumin (i.e., “esterified curcumin”). In some embodiments, the esterified form of curcumin may be fully esterified and have the following structure:

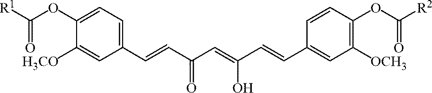

In Formula (1) above, R¹ and R² may be the same or different and are independently selected from hydrocarbon groups, more typically linear or branched alkyl or alkenyl groups, containing 1, 2, 3, 4, 5, 6, 7, 8, 9, 10, 11, 12, 13, 14, 15, 16, 17, 18, 19, or 20 carbon atoms, or a number of carbon atoms within a range bounded by any two of the foregoing values (e.g., 1-20, 2-20, 3-20, 4-20, 1-12, 2-12, 3-12, 4-12, 1-6, 2-6, 3-6, or 4-6 carbon atoms). In some embodiments, the esterified form of curcumin is partially esterified, in which case one of the C(O)R¹ or C(O)R² groups in Formula (1) is replaced with an H atom. Some particular examples of curcumin esters include curcumin diacetate, curcumin dipropanoate, curcumin dibutanoate, curcumin dipentanoate, curcumin dihexanoate, curcumin dioctanoate, curcumin didecanoate, curcumin didodecanoate, curcumin ditetradecanoate, curcumin dihexadecanoate, curcumin dioctadecanoate, curcumin monoacetate, curcumin monopropanoate, curcumin monobutanoate, curcumin monopentanoate, curcumin monohexanoate, curcumin monooctanoate, curcumin monodecanoate, curcumin monododecanoate, curcumin monotetradecanoate, curcumin monohexadecanoate, curcumin monooctadecanoate, and combinations thereof

In a second set of embodiments, the esterified natural product is an esterified form of catechin (i.e., “esterified catechin”). In some embodiments, the esterified form of catechin may be fully esterified and have the following structure:

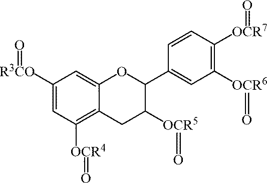

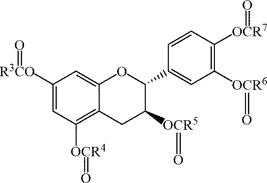

In Formula (2) or (2a) above, R³, R⁴, R⁵, R⁶, and R⁷ may be the same or different and are independently selected from hydrocarbon groups, more typically linear or branched alkyl or alkenyl groups, containing 1, 2, 3, 4, 5, 6, 7, 8, 9, 10, 11, 12, 13, 14, 15, 16, 17, 18, 19, or 20 carbon atoms, or a number of carbon atoms within a range bounded by any two of the foregoing values (e.g., 1-20, 2-20, 3-20, 4-20, 1-12, 2-12, 3-12, 4-12, 1-6, 2-6, 3-6, or 4-6 carbon atoms). In some embodiments, the esterified form of catechin is partially esterified, in which case one, two, three, or four of the C(O)R³, C(O)R⁴, C(O)R⁵, C(O)R⁶, and C(O)R⁷ groups in Formula (2) or (2a) is replaced with the respective number of H atoms. Some particular examples of catechin esters include catechin pentaacetate, catechin pentapropionate, catechin pentabutanoate, catechin tetrahexanoate, catechin tetraacetate, catechin tetrapropionate, catechin tetrabutanoate, catechin tetrahexanoate, catechin triacetate, catechin tripropionate, catechin tributanoate, catechin trihexanoate, catechin diacetate, catechin dipropionate, catechin dibutanoate, catechin dihexanoate, catechin monoacetate, catechin monopropionate, catechin monobutanoate, and catechin monohexanoate.

In a third set of embodiments, the esterified natural product is an esterified form of ascorbic acid (i.e., “esterified ascorbic acid” or “ascorbyl ester”). In some embodiments, the esterified form of ascorbic acid may be fully esterified and have the following structure:

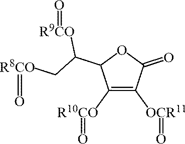

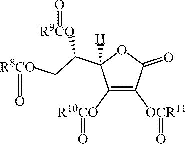

In Formula (3) or (3a) above, R⁸, R⁹, R¹⁰, and R¹¹ may be the same or different and are independently selected from hydrocarbon groups, more typically linear or branched alkyl or alkenyl groups, containing 1, 2, 3, 4, 5, 6, 7, 8, 9, 10, 11, 12, 13, 14, 15, 16, 17, 18, 19, or 20 carbon atoms, or a number of carbon atoms within a range bounded by any two of the foregoing values (e.g., 1-20, 2-20, 3-20, 4-20, 1-12, 2-12, 3-12, 4-12, 1-6, 2-6, 3-6, or 4-6 carbon atoms). In some embodiments, the esterified form of ascorbic acid is partially esterified, in which case one, two, or three of the C(O)R⁸, C(O)R⁹, C(O)R¹⁰, and C(O)R¹¹ groups in Formula (3) or (3a) is replaced with the respective number of H atoms. Some particular examples of ascorbyl esters include ascorbyl tetraacetate, ascorbyl tetrapropionate, ascorbyl tetrabutanoate, ascorbyl tetrahexanoate, ascorbyl triacetate, ascorbyl tripropionate, ascorbyl tributanoate, ascorbyl trihexanoate, ascorbyl diacetate, ascorbyl dipropionate, ascorbyl dibutanoate, ascorbyl dihexanoate, ascorbyl monoacetate, ascorbyl monopropionate, ascorbyl monobutanoate, and ascorbyl monohexanoate.

The esterified natural product (e.g., esterified form of curcumin, catechin, and/or ascorbic acid) and ionic iron can be included in the iron-containing composition in any suitable molar ratio, such as a molar ratio of 1000:1 to 1:1, or more particularly, a molar ratio of 1000:1, 900:1, 800:1, 700:1, 600:1, 500:1, 400:1, 300:1, 200:1, 100:1, 50:1, 20:1, 10:1, 5:1, 4:1, 3:1, 2:1, or 1:1, or a molar ratio within a range bounded by any two of the foregoing values.

In some embodiments, the composition further includes a co-solvent. The co-solvent can be any non-toxic solvent (typically, other than water), such as an alcohol or diol, such as ethanol or propylene glycol. The co-solvent is typically fully soluble in the edible oil and may be included in a concentration of precisely, about, or at least, for example, 0.1, 0.2, 0.5, 1, 2, 5, 10, 20, 30, 40, 50, 60, 70, 80, or 90 wt% (or vol%) or a concentration within a range bounded by any two of the foregoing values (e.g., 0.1-90 wt%, 0.2-90 wt%, 0.5 -90 wt%, 1-90 wt%, 2-90 wt%, 0.1-80 wt%, 0.2-80 wt%, 0.5 -80 wt%, 1-80 wt%, 2-80 wt%, 0.1-70 wt%, 0.2-70 wt%, 0.5 -70 wt%, 1-70 wt%, 2-70 wt%, 0.1-60 wt%, 0.2-60 wt%, 0.5 -60 wt%, 1-60 wt%, 2-60 wt%, 0.1-50 wt%, 0.2-50 wt%, 0.5 -50 wt%, 1-50 wt%, 2-50 wt%, 0.1-40 wt%, 0.2-40 wt%, 0.5 -40 wt%, 1-40 wt%, 2-40 wt%, 0.1-30 wt%, 0.2-30 wt%, 0.5 -30 wt%, 1-30 wt%, 2-30 wt%, 0.1-20 wt%, 0.2-20 wt%, 0.5 -20 wt%, 1-20 wt%, 2-20 wt%, 0.1-10 wt%, 0.2-10 wt%, 0.5 -10 wt%, 1-10 wt%, 2-10 wt%, 0.1-5 wt%, 0.2-5 wt%, 0.5 -5 wt%, 1-5 wt%, or 2-5 wt%).

In some embodiments, the esterified natural product (e.g., esterified form of curcumin, catechin, and/or ascorbic acid) is included in a concentration of precisely, at least, or about, for example, 0.01 mg/mL, 0.02 mg/mL, 0.03 mg/mL, 0.04 mg/mL, 0.05 mg/mL, 0.1 mg/mL, 0.15 mg/mL, 0.2 mg/mL, or 0.5 mg/mL (or 0.001 wt%, 0.002 wt%, 0.003 wt%, 0.004 wt%, 0.005 wt%,0.01 wt%, 0.02 wt%, 0.03 wt%, 0.04 wt%, 0.05 wt%, 0.1 wt%, 0.15 wt%, 0.2 wt%, or 0.5 wt%), or a concentration within a range bounded by any two of the foregoing values (e.g., 0.01-0.5 mg/mL, or 0.01-0.5 wt%).

In some embodiments, the ionic iron is included in a concentration of precisely, at least, or about, for example, 0.001 mg/mL, 0.002 mg/mL, 0.003 mg/mL, 0.004 mg/mL, 0.005 mg/mL, 0.01 mg/mL, 0.02 mg/mL, 0.03 mg/mL, 0.04 mg/mL, 0.05 mg/mL, or 0.1 mg/mL (or 0.0001 wt%, 0.0002 wt%, 0.0003 wt%, 0.0004 wt%, 0.0005 wt%, 0.001 wt%, 0.002 wt%, 0.003 wt%, 0.004 wt%, 0.005 wt%, 0.01 wt%, 0.02 wt%, 0.03 wt%, 0.04 wt%, 0.05 wt%, or 0.1 wt%), or a concentration within a range bounded by any two of the foregoing values (e.g., 0.001-0.1 mg/mL, or 0.001-0.1 wt%).

In another aspect, the present disclosure is directed to methods of preparing the iron-containing compositions described above. In the methods, at least the esterified form of natural product, the edible oil, and ionic iron are combined, either in one step or in separate steps. A first method may include the following steps: combining the esterified form of natural product (e.g., an esterified form of curcumin, catechin, and/or ascorbic acid) and edible oil to form a mixture, and further combining the mixture with the ionic iron (or adding the ionic iron to the mixture) to form the iron-containing composition. The term “mixture”, as used herein, also includes the possibility of a solution in which the esterified natural product is fully dissolved in the oil. In some embodiments, the mixture of esterified natural product and oil (“Solution A”) is heated to a low temperature above room temperature (e.g., precisely, about, at least, or up to 30, 35, 40, 45, or 50° C., or a range between any two of these temperatures) for a period of precisely, about, or at least 5, 10, 15, 20, 25, 30, 35, 40, 45, 50, 55, or 60 minutes until the esterified natural product is substantially or completely dissolved in the oil. Solution A is typically cooled to room temperature (about 25° C.) before combining it with the ionic iron, wherein the ionic iron may be in solid (powder) form or in solution when combined with Solution A. A second method may include the following steps: (i) combining the esterified form of natural product (e.g., an esterified form of curcumin, catechin, and/or ascorbic acid) and edible oil to form a first mixture (i.e., Solution A); dispersing the ionic iron in a co-solvent (e.g., ethanol, propylene glycol, or other food grade co-solvent) to form a second mixture (i.e., Solution B); and combining the first and second mixtures (i.e., Solutions A and B) to form the iron-containing composition. The step of combining Solutions A and B can be performed by adding Solution A to Solution B, or vice-versa. The esterified forms of natural products can be prepared by means well known in the art, as further discussed in the Examples section. of this disclosure.

In another aspect, the present disclosure is directed to a food product containing any of the iron-containing compositions described above, wherein the iron-containing composition may be produced by any of the methods described above. The food product may be, for example, a grain, grain-containing product, grain-derived product, vegetable, vegetable-containing product, vegetable-derived product, meat, meat-containing product, or meat-derived product. The iron-containing composition can be incorporated into the food product by any of the means well known in the art, e.g., by blending (if applicable), injecting, or coating.

In yet another aspect, the present disclosure is directed to a method of treating an iron deficiency in a subject by administering a therapeutic amount of any one of the iron-containing compositions described above to the subject. The iron-containing composition may be administered by itself or in the form of a food product or beverage in which the iron-containing composition is incorporated. In certain embodiments, the subject being treated may have a lower than optimal or deficient blood iron level, red blood cell count (e.g., hematocrit), or hemoglobin. In some embodiments, the subject being treated has anemia. The anemia may be, for example, iron deficiency anemia, vitamin deficiency anemia, an anemia associated with bone marrow disease, an anemia associated with inflammation, hemolytic anemia, or sickle cell anemia.

Examples have been set forth below for the purpose of illustration and to describe the best mode of the invention at the present time. However, the scope of this invention is not to be in any way limited by the examples set forth herein.

### EXAMPLES

### General

Materials: butyric anhydride and 4-dimethylamino pyridine were obtained commercially with 98 and 99% purity, respectively. Curcumin and n-octanoic anhydride were obtained commercially with 97 and 95% purity, respectively. Silica gel (P60, 40-63 µm, 60 Å) and Silica Gel 60 F254 Coated Aluminum-Backed TLC Sheets were obtained from commercial sources.

Characterization: A 500 MHz NMR spectrometer was used for ¹H NMR (500 MHz) and ¹³C NMR (125 MHz) spectra in CDCl₃. Fourier transform infrared spectra (ATR-FTIR) were recorded on a Shimadzu IRAffinity-1S spectrophotometer. UV-Vis was recorded on a Shimadzu UV-2600 spectrophotometer.

Synthesis of curcumin dibutanoate. To a round bottom flask with a magnetic stir bar, septa, and nitrogen inlet, 300 mg (0.815 mmol) of curcumin, 0.8 mL (4.9 mmol) of butyric anhydride, 25 mg (0.2 mmol) of 4-dimethylamino pyridine, and 8 mL of acetonitrile were added. The reaction mixture was stirred at room temperature overnight under a nitrogen blanket. The progress of the reaction was followed by thin-layer chromatography (TLC). After completing the reaction, 40 mL of ethyl acetate was added to the reaction mixture and the organic phase was washed with (4 × 20 mL) of HCl (2 M). After that, the organic phase was washed with (3 × 20 mL) of NaHCO₃ (0.4 M). Then, the organic solvent was dried with sodium sulfate and evaporated by a rotary evaporator. Finally, the crude product was washed with cold hexanes to obtain the pure product as a yellow solid in 65% yield (270 mg).

Synthesis of curcumin dioctanoate. To a round bottom flask with a magnetic stir bar, septa, and nitrogen inlet, 300 mg (0.815 mmol) of curcumin, 1.6 mL (5.1 mmol) of octanoic anhydride, 25 mg (0.2 mmol) of 4-dimethylamino pyridine, and 9 mL of acetonitrile were added. The reaction mixture was stirred at room temperature overnight under a nitrogen blanket. The progress of the reaction was followed by thin-layer chromatography (TLC). After completing the reaction, 60 mL of ethyl acetate was added to the reaction mixture and the organic phase was washed with (4 × 20 mL) of HCl (2 M). After that, the organic phase was washed with (3 × 20 mL) of NaHCO₃ (0.4 M). Then, the organic solvent was dried with sodium sulfate and evaporated by a rotary evaporator. Finally, the crude product was washed with hexanes to obtain the pure product as a light-yellow solid in 77% yield (390 mg).

Preparation of curcumin in coconut oil. To a round bottom flask with a magnetic stir bar, 20 mg of curcumin and 25 mL (23000 mg) of coconut oil were added. The flask was placed in a water bath at 40° C. and the mixture was stirred for 30 min. The sample was then cooled to room temperature and centrifuged to remove non-dissolved residue of curcumin. The concentration of curcumin in coconut oil was around 0.08 wt %.

Preparation of curcumin dibutanoate in coconut oil. To a round bottom flask with a magnetic stir bar, 20 mg of curcumin dibutanoate and 3 mL (2760 mg) of coconut oil were added. The flask was placed in a water bath at 40° C. and the mixture was stirred until curcumin dibutanoate was completely dissolved in the coconut oil (around 20 min). The sample was then cooled to room temperature and used for the next studies. Throughout storage, the curcumin dibutanoate in coconut oil remained clear without any precipitation. The concentration of curcumin dibutanoate in coconut oil was 0.72 wt % (6.7 mg/mL).

Preparation of curcumin dioctanoate in coconut oil. To a round bottom flask with a magnetic stir bar, 20 mg of curcumin dioctanoate and 10 mL (9200 mg) of coconut oil were added. The flask was placed in a water bath at 50° C. and the mixture was stirred until curcumin dibutanoate was completely dissolved in the coconut oil (around 20 min). The sample was then cooled to room temperature and used for the next studies. The concentration of curcumin dioctanoate in coconut oil was 0.22 wt % (2 mg/mL).

Preparation of curcumin dibutanoate in canola oil. To a round bottom flask with a magnetic stir bar, 20 mg of curcumin dibutanoate and 5 mL (4600 mg) of coconut oil were added. The flask was placed in a water bath at 45° C. and the mixture was stirred until curcumin dibutanoate was completely dissolved in the coconut oil. The sample was then cooled to room temperature and used for the next studies. Throughout storage, the curcumin dibutanoate in coconut oil remained clear. The concentration of curcumin dibutanoate in coconut oil was 0.43 wt% (4 mg/mL).

Preparation of curcumin dibutanoate complex with FeCl₂ in coconut oil. To a culture tube containing 4 mg of FeCl₂•4H₂O, 3.5 mL of curcumin dibutanoate solution in coconut oil (5.7 mg/mL) was added. Then the mixture was vortexed for 30 min at room temperature. The sample was centrifuged to remove non-dissolved residue of FeCl₂.

Preparation of curcumin dioctanoate complex with FeCl₂ in coconut oil. To a culture tube containing 3 mg of FeCl₂•4H₂O, 10 mL of curcumin dioctanoate solution in coconut oil (2 mg/mL) was added. Then the mixture was vortexed for 30 min at room temperature. The sample was centrifuged to remove non-dissolved residue of FeCl₂.

Preparation of curcumin dibutanoate complex with FeCl₂ in coconut oil by using ethanol as a cosolvent. To a culture tube containing 4 mg of FeCl₂•4H₂O and 0.08 mL of ethanol, 3.5 mL of curcumin dibutanoate solution in coconut oil (5.7 mg/mL) was added. Then the mixture was vortexed for 5 min at room temperature.

Preparation of curcumin dioctanoate complex with FeCl₂ in coconut oil by using ethanol as a cosolvent. To a culture tube containing 3 mg of FeCl₂•4H₂O and 0.2 mL of ethanol, 10 mL of curcumin dioctanoate solution in coconut oil (2 mg/mL) was added. Then the mixture was vortexed for 5 min at room temperature.

Preparation of curcumin dibutanoate complex with FeCl₂ in canola oil by using ethanol as a cosolvent. To a culture tube containing 4 mg of FeCl₂•4H₂O and 0.15 mL of ethanol, 5 mL of curcumin dibutanoate solution in canola oil (4 mg/mL) was added. Then the mixture was vortexed for 5 min at room temperature.

In this example, the stability of curcumin was increased by a modification to small chain and medium-chain fatty ester derivatives. For this purpose, curcumin dibutanoate (small chain fatty ester) and curcumin dioctanoate (medium-chain fatty ester) were synthesized. As shown in the scheme below, curcumin dibutanoate and curcumin dioctanoate were synthesized by using curcumin in the presence of butyric anhydride and n-octanoic anhydride respectively. In these reactions, 4-dimethylaminopyridine (DMAP) was used as a catalyst and the reactions were carried out in acetonitrile at room temperature.

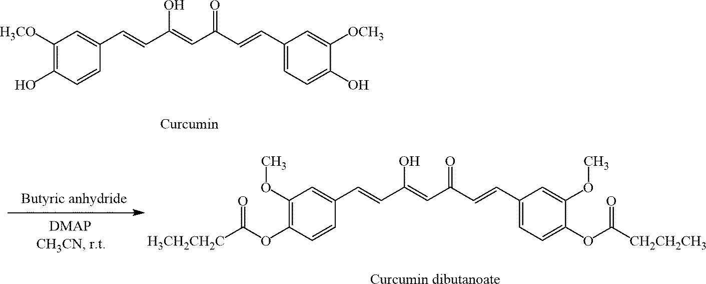

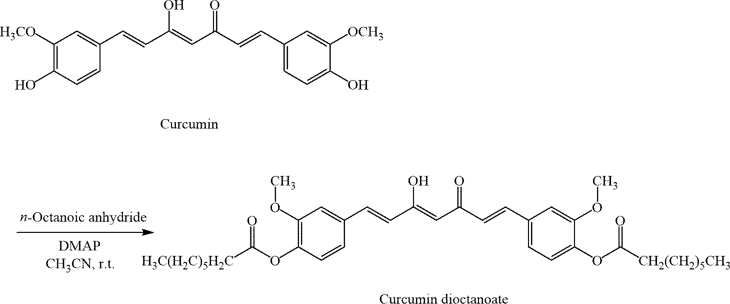

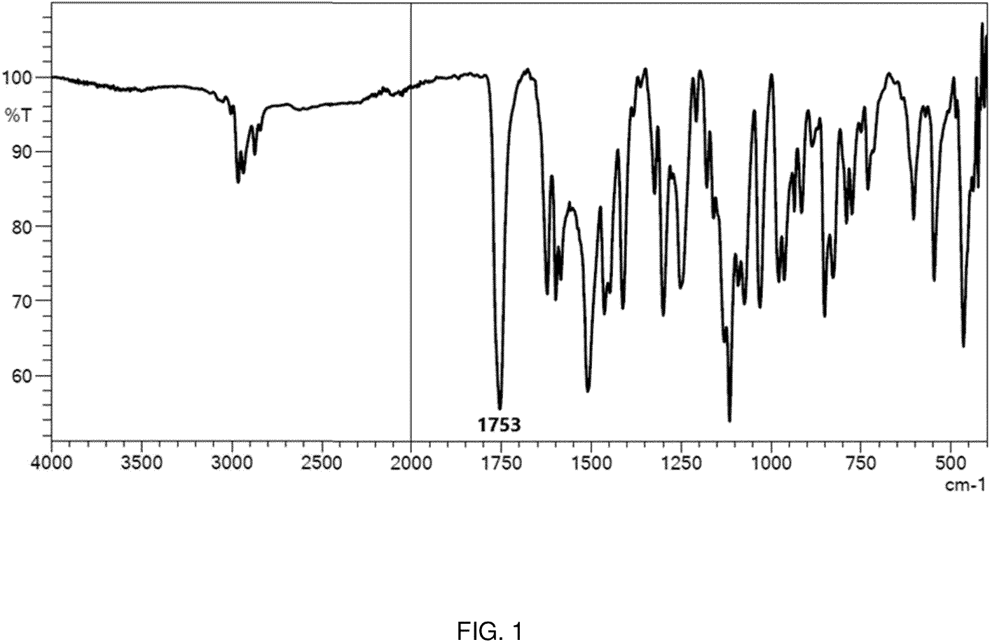

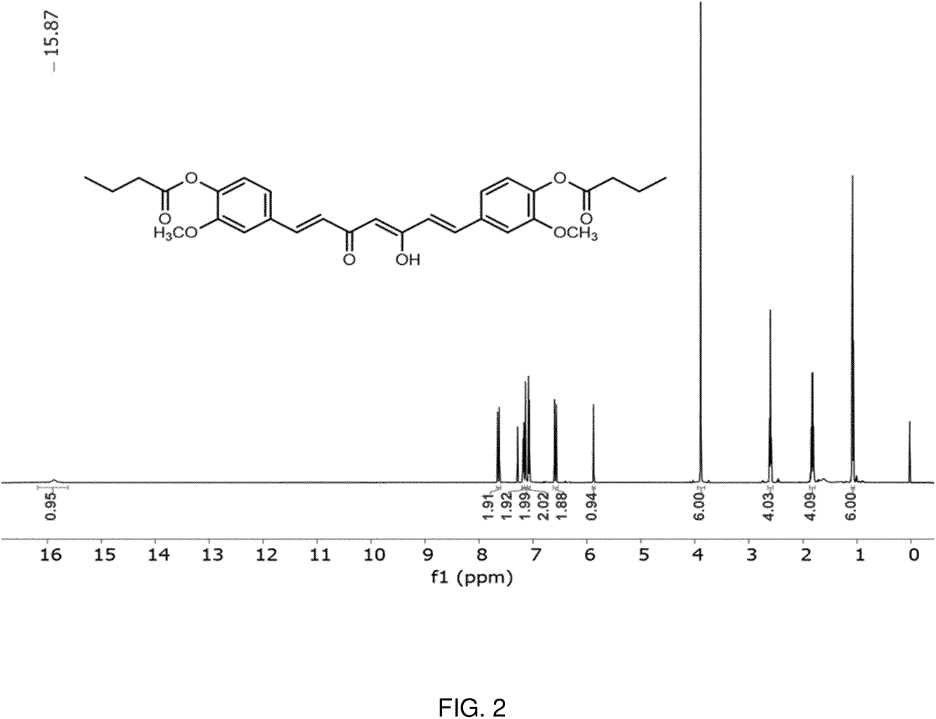

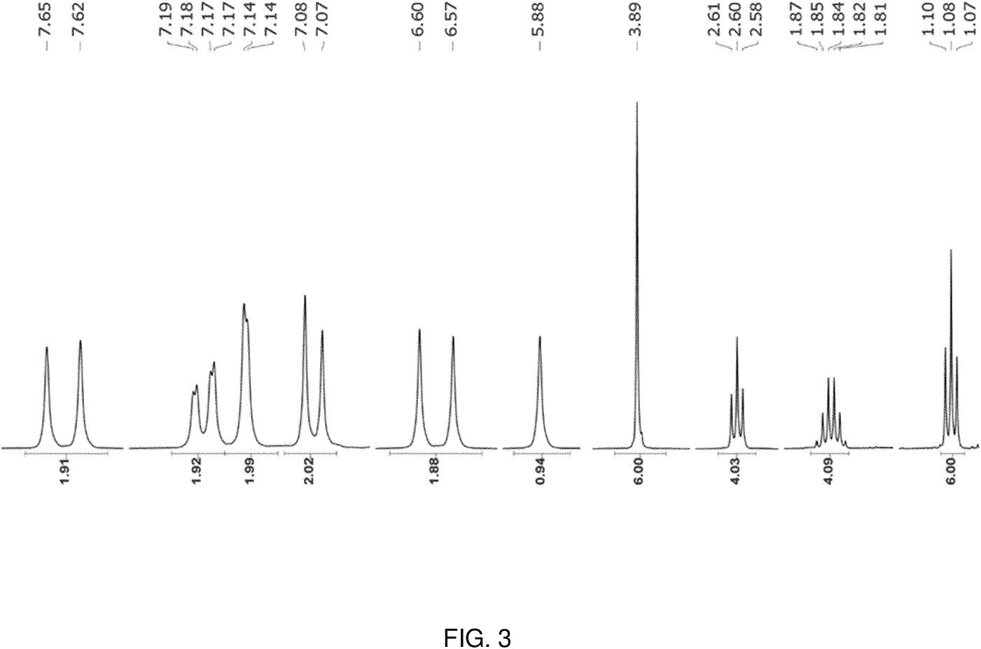

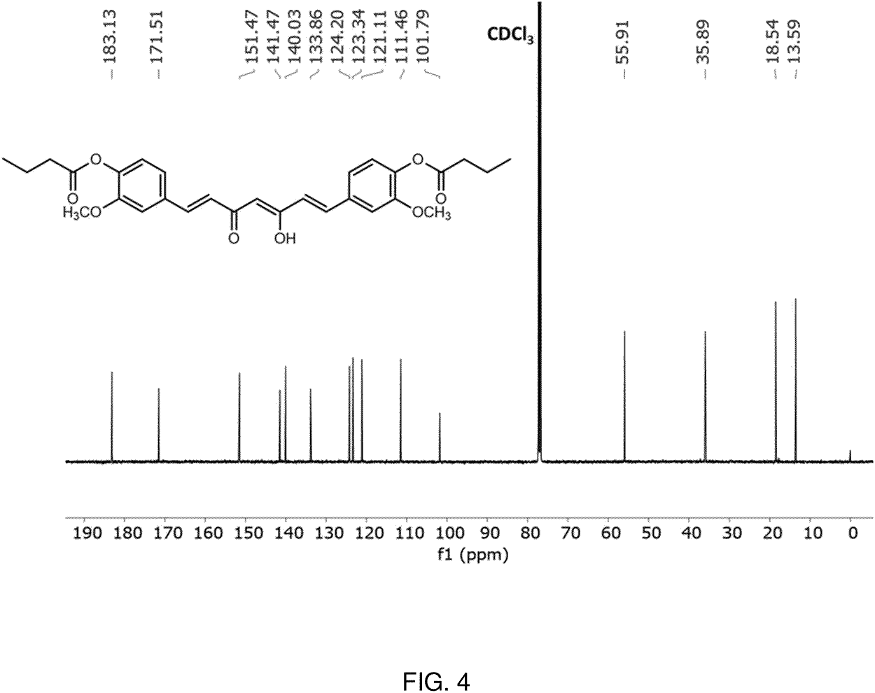

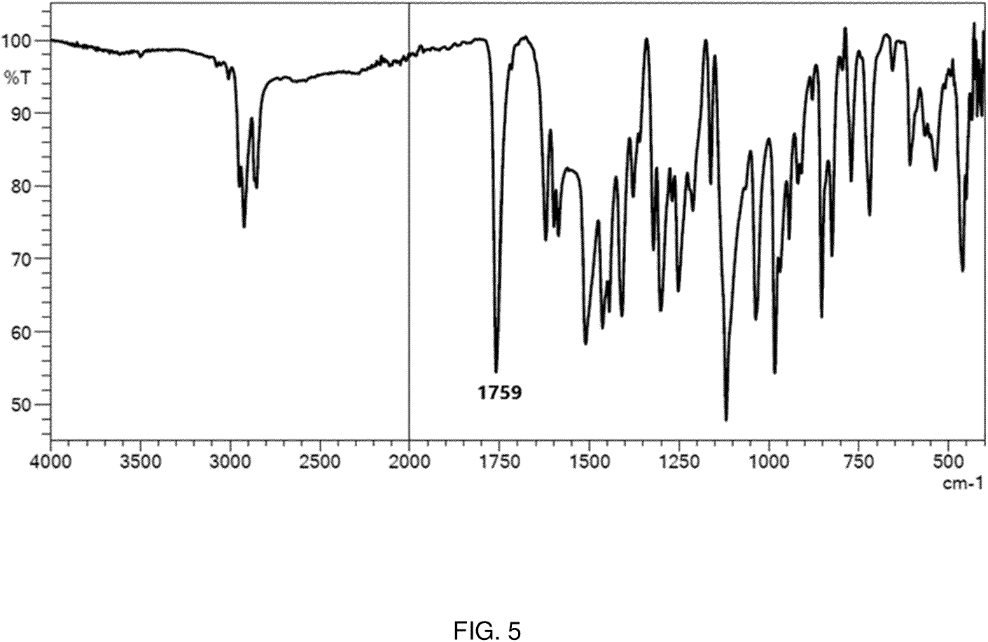

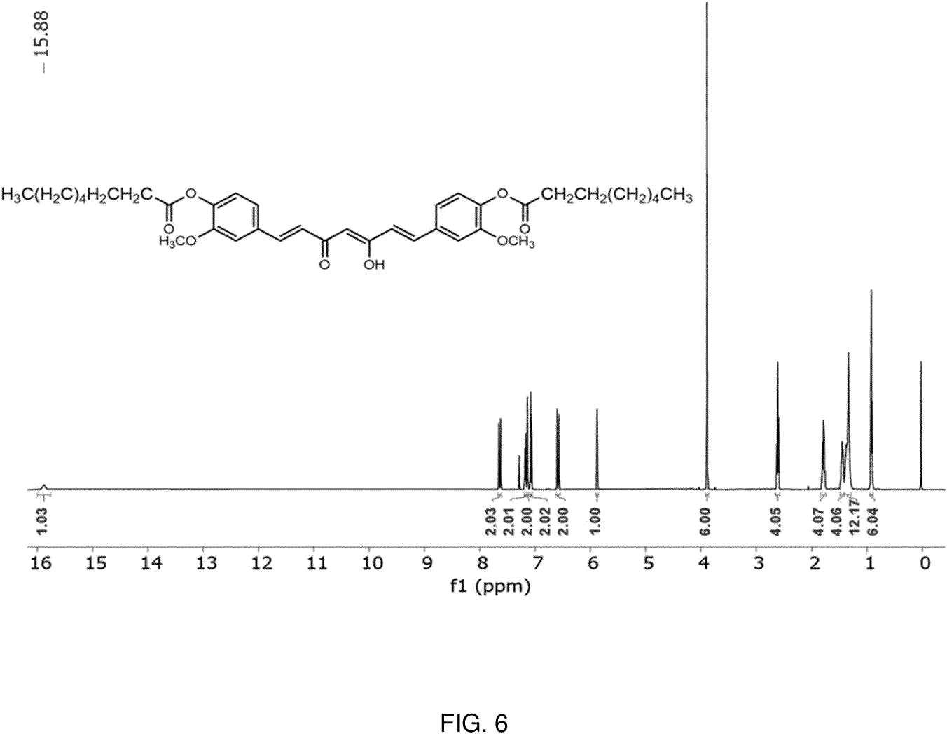

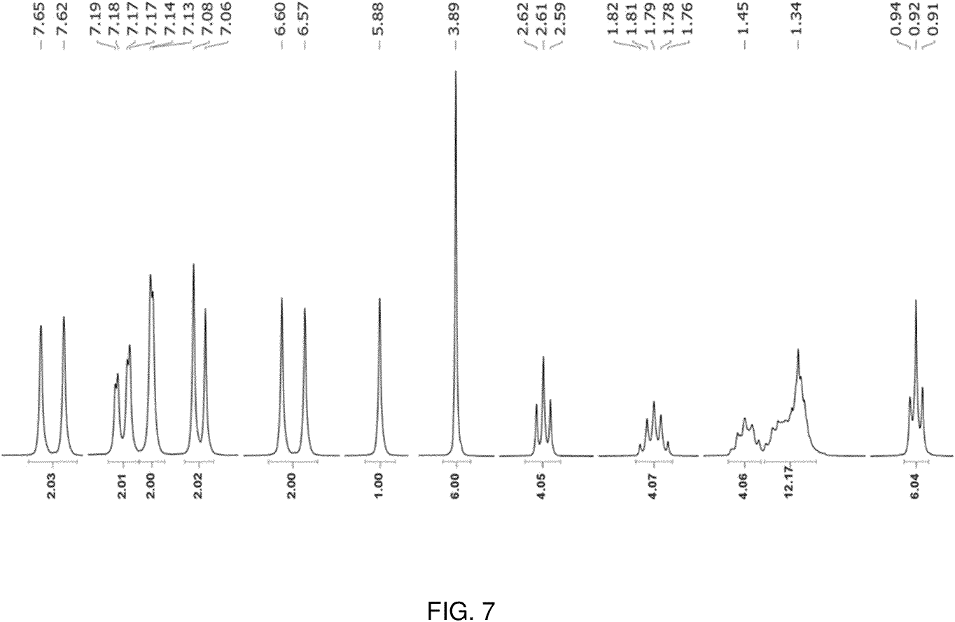

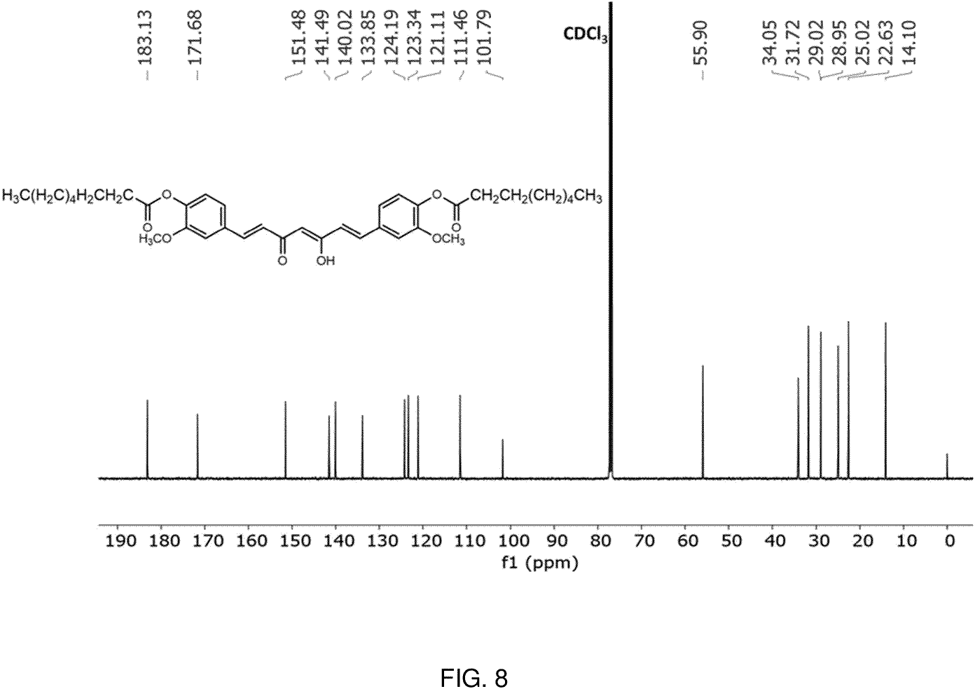

The yield of each compound was reported as the isolated pure product. The pure curcumin dibutanoate and curcumin dioctanoate were characterized by FTIR, ¹H NMR, and ¹³C NMR (FIGS. 1-8).

After modification of curcumin to curcumin dibutanoate and curcumin dioctanoate, the stability of these compounds significantly increased so that these compounds could be dissolved in oil, whereas the pure curcumin (97% purity) needed to be kept below 0° C. Most oxidation and auto-oxidation of curcumin take place on the phenolic hydroxyl groups (M. Salem et al., RSC Advances 2014;4:10815-29). Therefore, by conversion of these functional groups to the corresponding ester forms, the rate of curcumin degradation decreases.

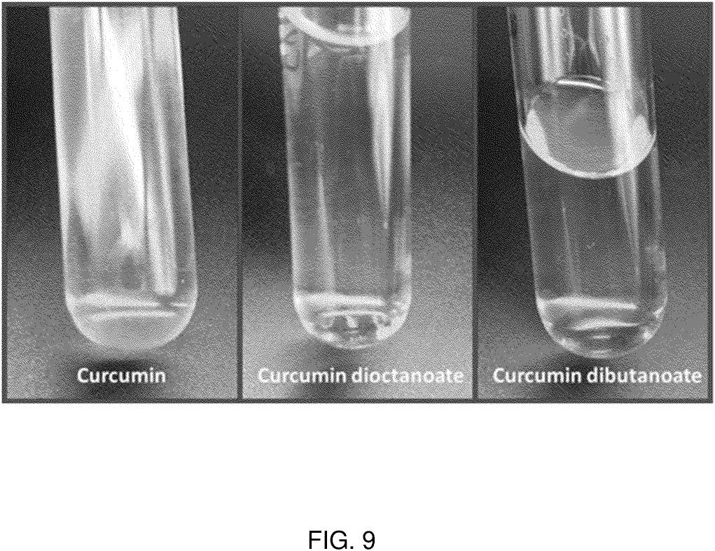

The oil solubility of curcumin increased by a modification to these ester derivatives so that 0.72 wt% curcumin dibutanoate could be dissolved in coconut oil (6.7 mg/mL). The maximum solubility of curcumin dioctanoate in coconut oil was around 0.22 wt % (2 mg/mL) and this amount was around 0.08 wt% for pure curcumin (FIG. 9). FIG. 9 shows the solubility of curcumin, curcumin dibutanoate, and curcumin dioctanoate in coconut oil. Among these compounds, curcumin dibutanoate exhibited the best result of solubility in coconut oil. Curcumin dibutanoate could be dissolved in canola oil as a long chain triglyceride. The maximum solubility of curcumin dibutanoate in canola oil was 0.43 wt% (4 mg/mL).

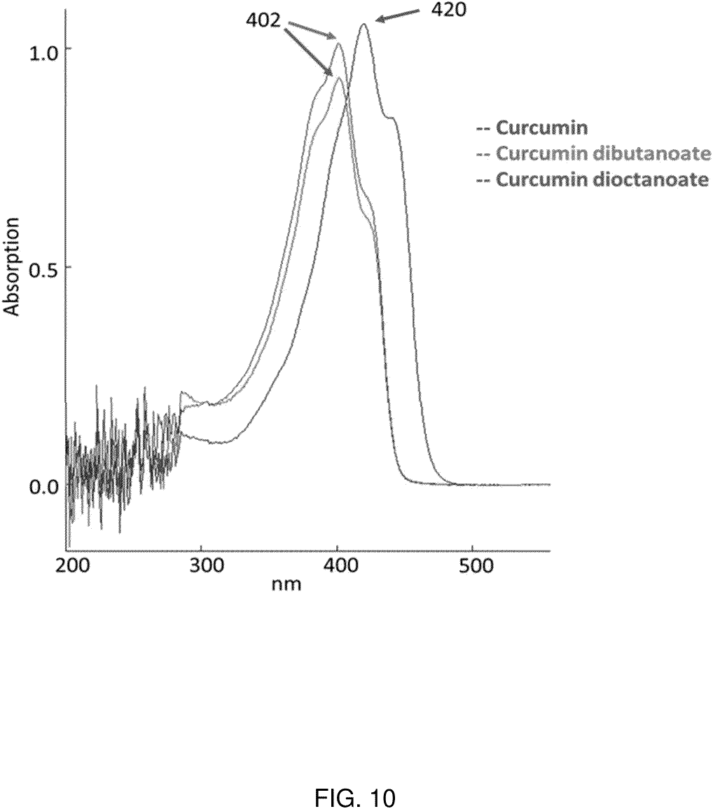

FIG. 10 shows UV-Vis absorption spectra of dissolved curcumin, curcumin dibutanoate, and curcumin dioctanoate in coconut oil. As the spectra displays the λₘₐₓ of curcumin dibutanoate, and curcumin dioctanoate in coconut oil is 402 nm while the dissolved curcumin in coconut oil shows the λₘₐₓ 420 nm. This hypsochromic shift indicates that by esterification of hydroxyl groups of curcumin, the resonance between lone pair electron on phenolic hydroxyls and aromatic rings decrease.

One of the best aspects of curcumin dibutanoate can appear during the digestion of this compound. Curcumin dibutanoate can be enzymatically hydrolyzed by lipase in the intestinal phase and curcumin and butyric acid can be produced. Butyric acid is one of the main components of short-chain fatty acids (SCFAs), used as a source of energy for the epithelial cells to improve the intestinal barrier for avoiding the translocation of antigens and pathogens (C. A. Morais et al., The Journal of Nutritional Biochemistry 2016;33:1-7). Moreover, butyric acid increases the population of bacteria acidified the intestinal pH and subsequently inhibits the growth of harmful pathogens. Furthermore, butyric acid increases the villus surface area and improves the functionality of the duodenal brush border membrane to enhance the bioavailability and absorption of essential nutrients and keep the intestine intact and healthy (T. Hou et al., Nutrients, 2018, 10:418).

Because curcumin dibutanoate is a substantially hydrophobic molecule, most of its digestion occurs in the intestinal phase. Therefore, the possibility of side reactions leading to degradation of the released curcumin is minimized and consequently can improve the bioavailability of curcumin. Recent studies show that curcumin has a valuable effect on the gut microbiota by increasing the growth of beneficial bacteria and limiting the pathogenic ones (T. Hou et al., Ibid.). By digestion of curcumin dibutanoate and in situ formation of curcumin in the intestine, this beneficial effect on the gut microbiota community can increase.

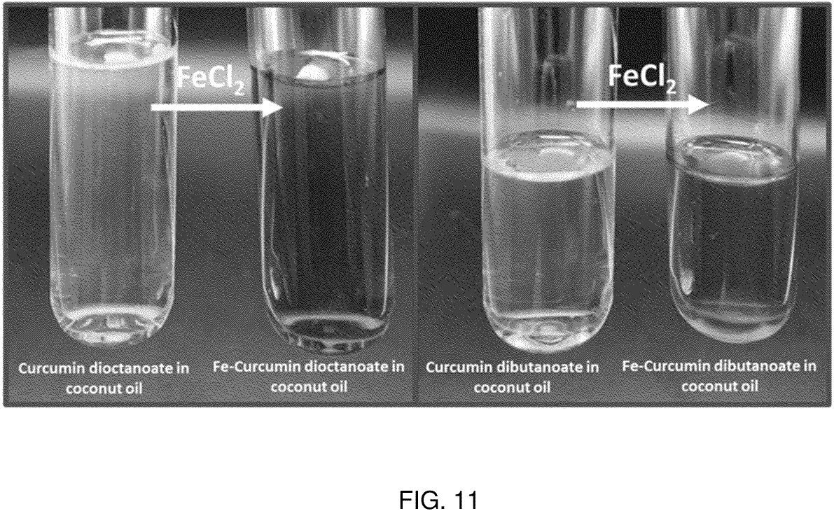

Finally, a soluble complex with FeCl₂•4H₂O and curcumin dibutanoate and curcumin dioctanoate in an oil phase was prepared. It should be mentioned that curcumin has a beta-diketone group in its structure that mostly tautomerizes to keto-enol form. Therefore, curcumin can act as a ligand to form a complex with a number of transition metal salts such as Fe, Cu, Ni, Mn, and Zn (M. Refat, Spectrochimica Acta Part A: Molecular and Biomolecular Spectroscopy 2013;105:326-37). In this work, the obtained results of the spectroscopy (FTIR, ¹H NMR, and ¹³C NMR) demonstrated that by modification of curcumin to curcumin dibutanoate and curcumin dioctanoate, the keto-enol group was intact. This means that these curcumin ester derivatives can form a complex with iron and makes it soluble in the edible oils. To demonstrate this concept, first, 3.5 mL of a solution of curcumin dibutanoate in coconut oil (5.7 mg/mL) was mixed with 4 mg of FeCl₂•4H₂O. The molar ratio between iron and curcumin dibutanoate was 1:2. The sample was centrifuged to remove non-dissolved residue of FeCl₂•4H₂O. The change of the oil color from yellow to light brown indicates the formation of a soluble complex between iron and curcumin dibutanoate (FIG. 11). This experiment was repeated for curcumin dioctanoate and the results were the same as for curcumin dibutanoate.

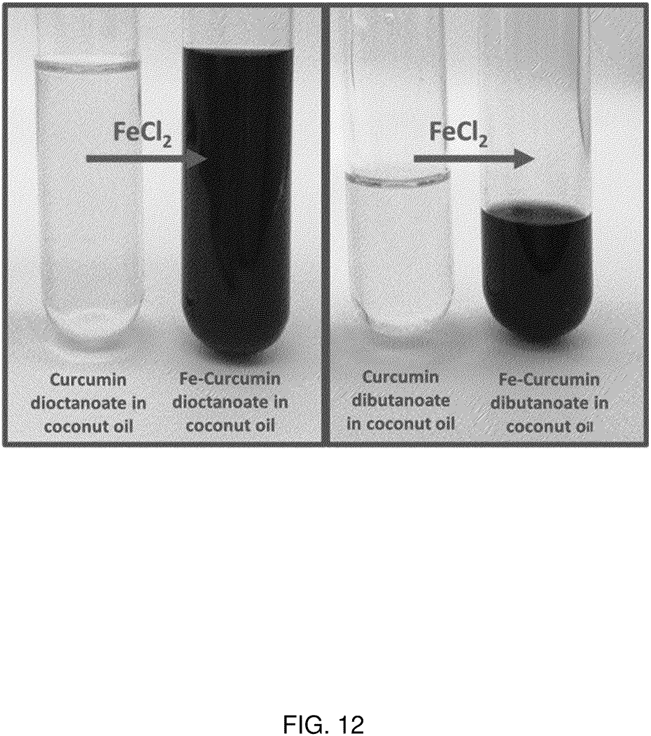

The presence of water in the structure of iron chloride may prevent the dispersion of this salt in oil, and consequently, the formation of an iron complex in the oil phase decreases. To overcome this problem, FeCl₂•4H₂O was dispersed in a least amount of ethanol and then added to the oil phase. The used ethanol was 2% of the total volume. By this modification, a black color immediately appeared. It means that most iron cations were coordinated by curcumin dibutanoate or curcumin dioctanoate in coconut oil (FIG. 12). Notably, both of these complexes were almost soluble in the oil phase.

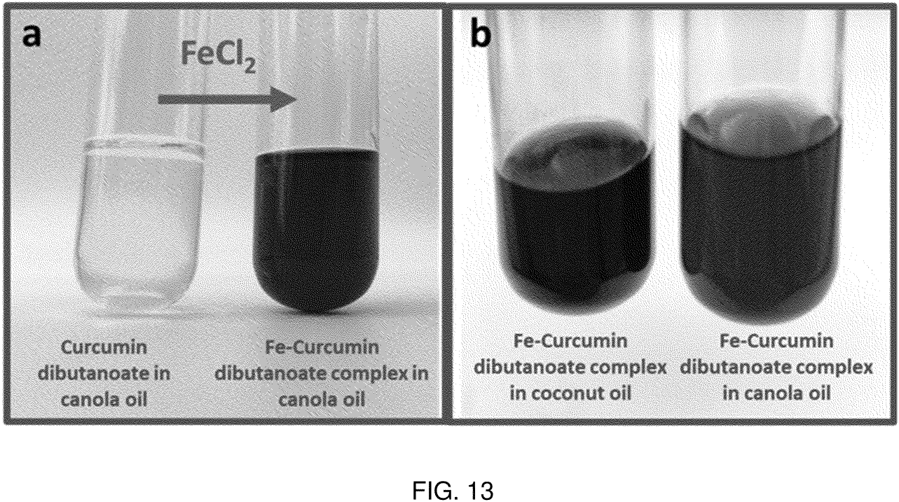

The solubility of FeCl₂ in canola oil by the formation of the curcumin dibutanoate complex with iron was also studied. The procedure was the same as dissolving FeCl₂ in coconut oil and using 2% of ethanol as a cosolvent (FIG. 13a). In comparison to the dissolved Fe-curcumin dibutanoate complex in coconut oil, the dissolved Fe-curcumin dibutanoate complex in canola oil was clearer and its color was dark brown (FIG. 13b).

In summary, curcumin dibutanoate and curcumin dioctanoate were synthesized as two curcumin ester derivatives. By this change, the solubility of curcumin increased in edible oils. Curcumin dibutanoate was nine times more soluble and curcumin dioctanoate was three times more soluble than curcumin in coconut oil. The stability of curcumin was increased by modification of curcumin to the corresponding ester derivatives. This parameter can improve the bioavailability of curcumin. These curcumin ester derivatives formed a complex with FeCl₂ and made iron soluble in oil. The oil-soluble complex of iron with curcumin dioctanoate can improve Fe delivery specially in anemia cases. Moreover, the presence of butanoate groups in this complex can be hydrolyzed to butyric acid that is beneficial for gut microbiota and the health of the intestine.

### Synthesis of Catechin-penta Butanoate

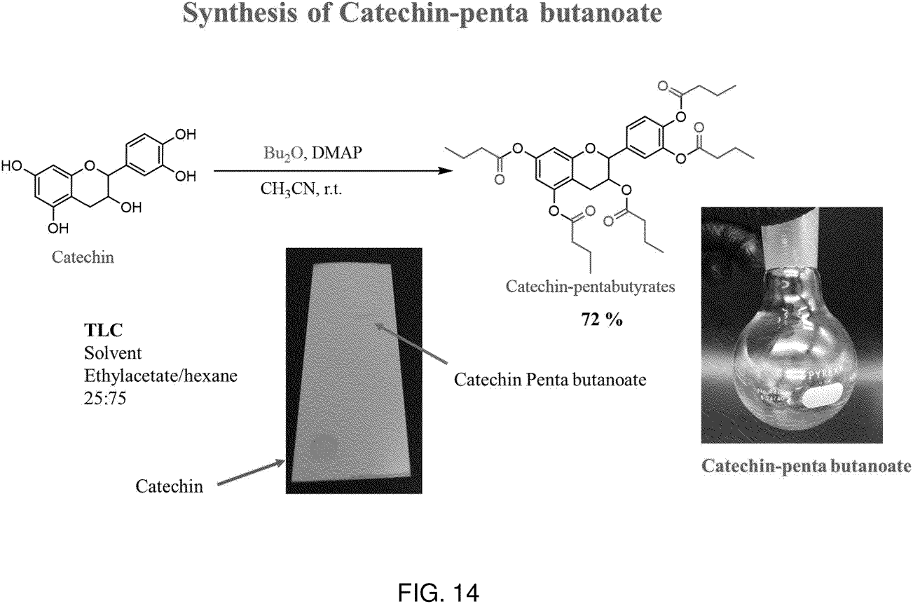

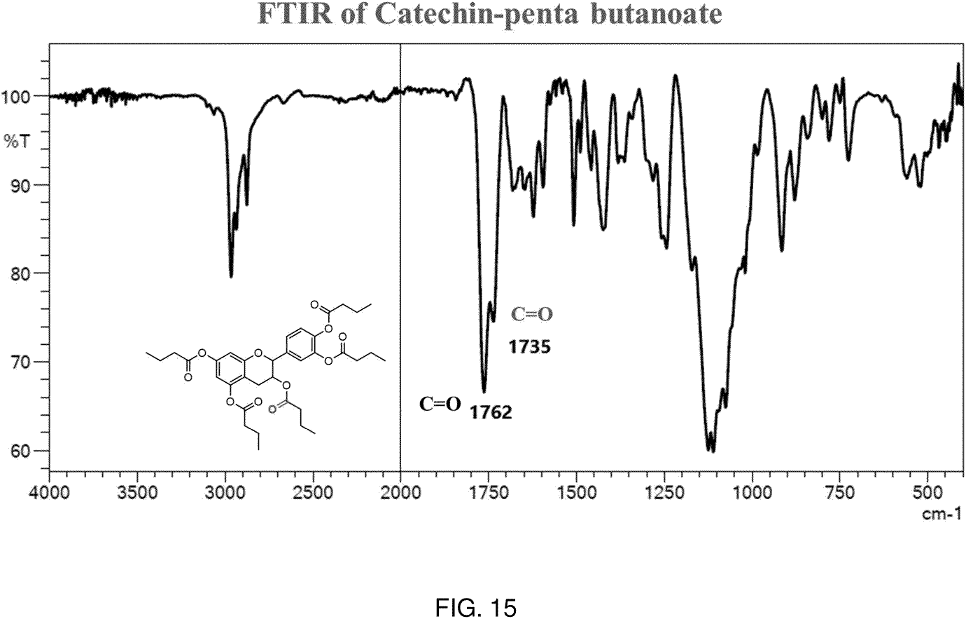

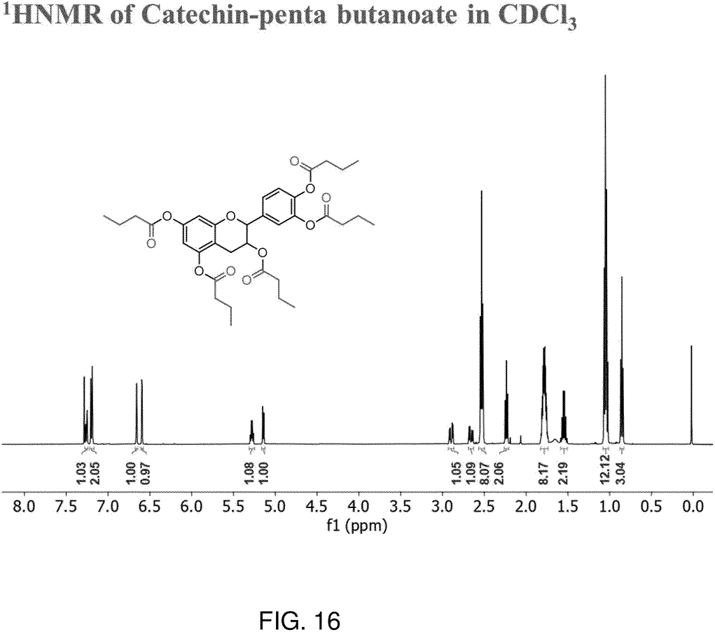

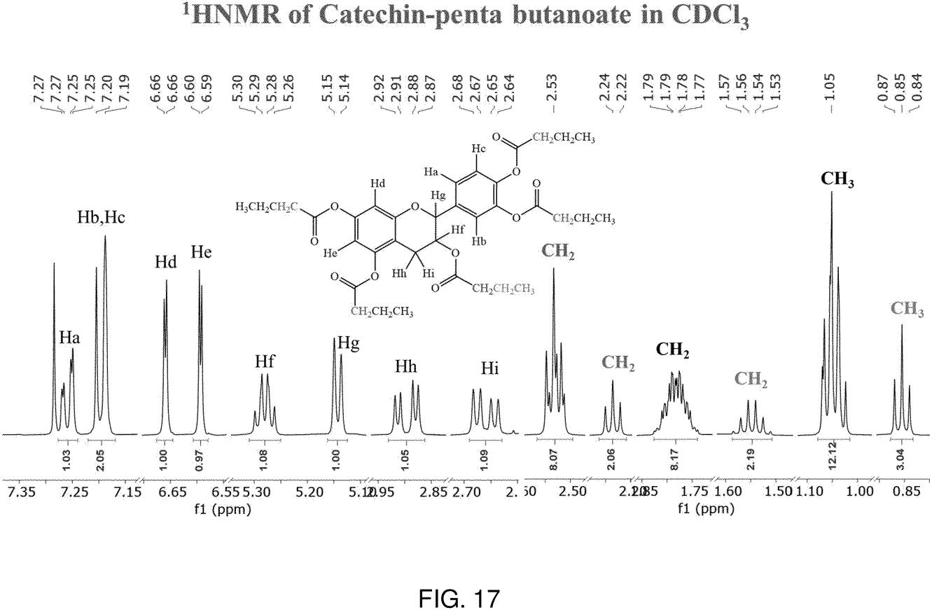

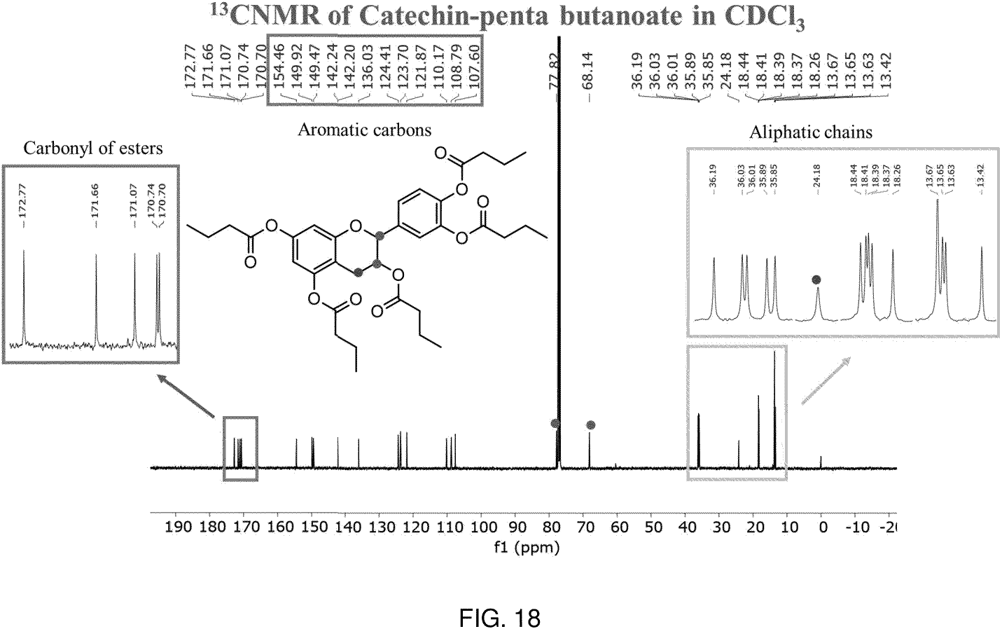

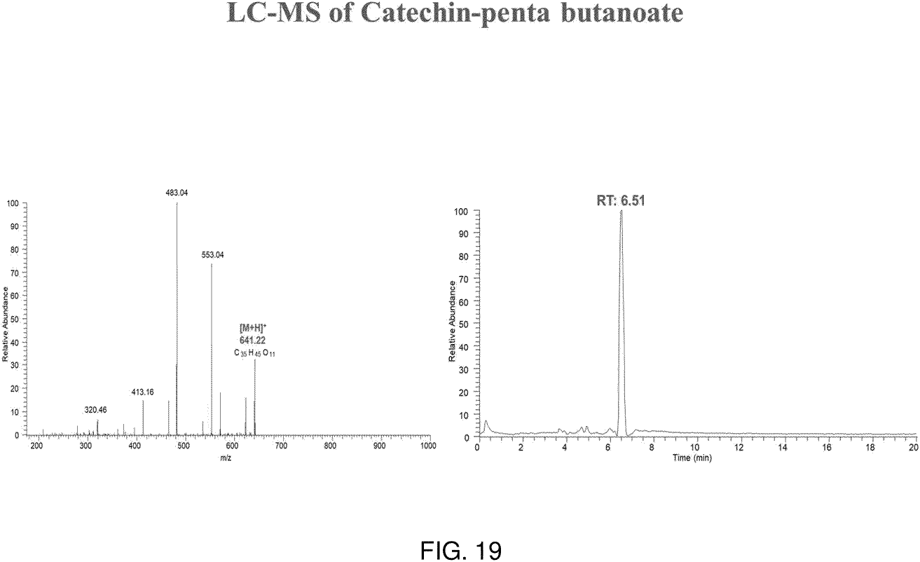

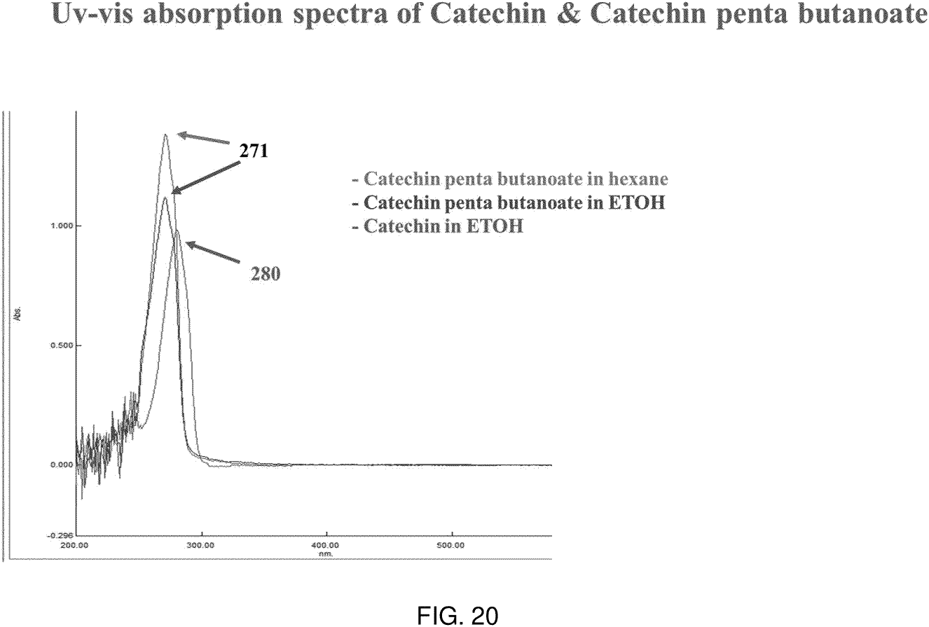

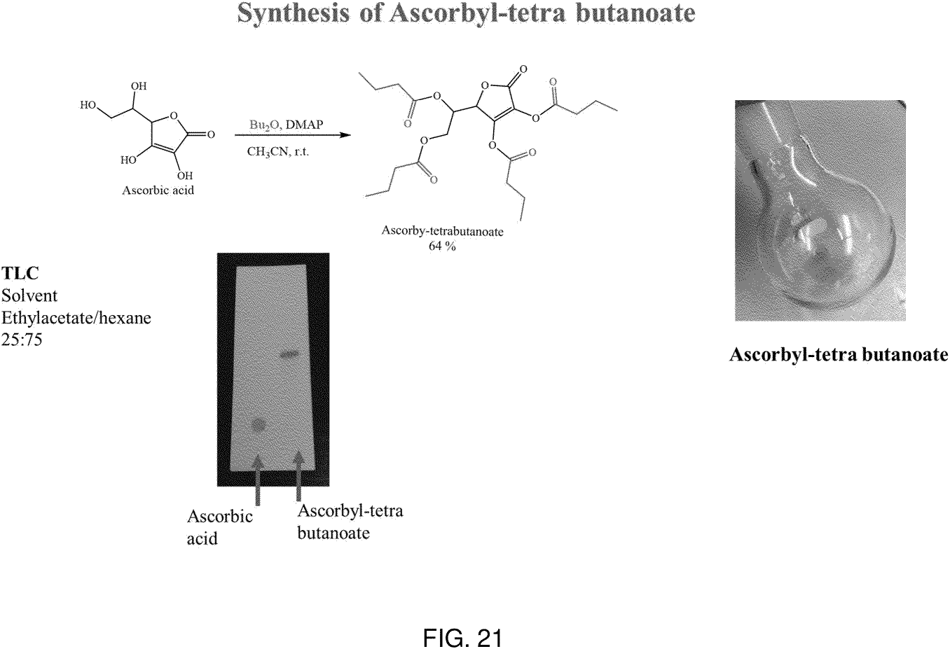

To a round bottom flask with a magnetic stir bar, septa, and nitrogen inlet, 200 mg (0.70 mmol) of catechin. H₂O, 2.06 mL (12.6 mmol) of butyric anhydride, 35 mg (0.28 mmol) of 4-dimethylamino pyridine, and 5 mL of acetonitrile were added. The reaction mixture was stirred at room temperature for 24 h under a nitrogen blanket. The progress of the reaction was followed by thin-layer chromatography (TLC). After completing the reaction, the excess amount of butyric anhydride was evaporated and the crude product was extracted with 30 mL of ethyl acetate. Then, the organic phase was washed with (4 × 20 mL) of HCl (2 M). After that, the organic phase was washed with (3 × 20 mL) of NaHCO₃ (0.4 M). Finally, the organic solvent was dried with sodium sulfate and evaporated by a rotary evaporator to obtain the product as a light-yellow viscose liquid in 81 % yield (337 mg). FIGS. 14-21 show preparation and characterization of catechin esters.

### Synthesis of Ascorbyl-tetra Butanoate

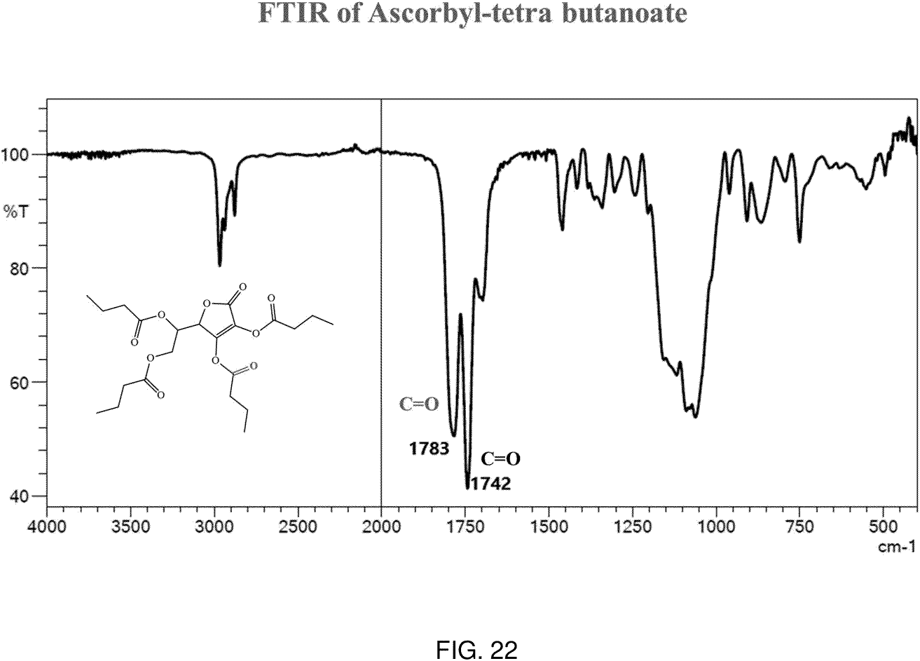

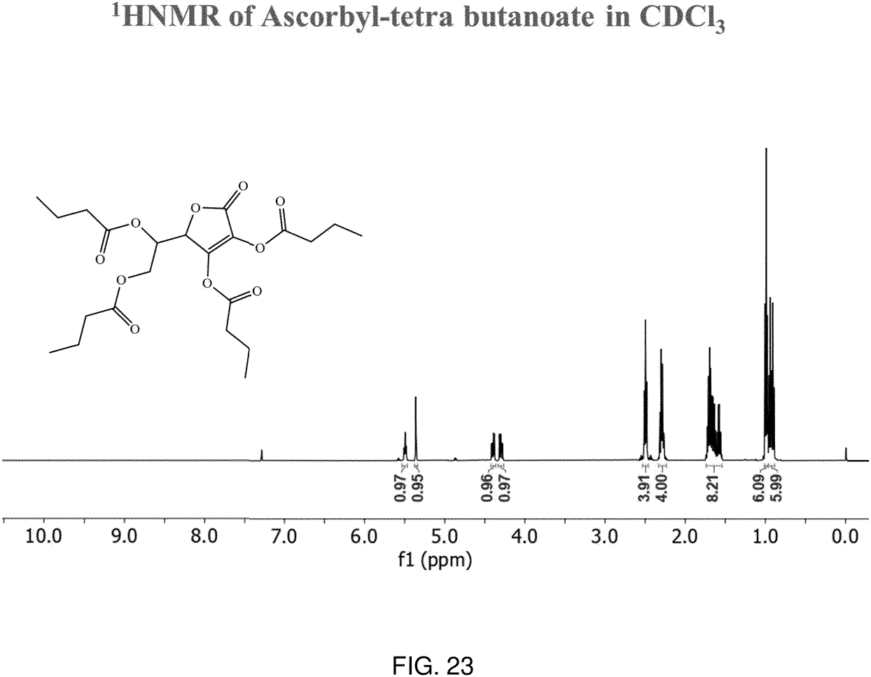

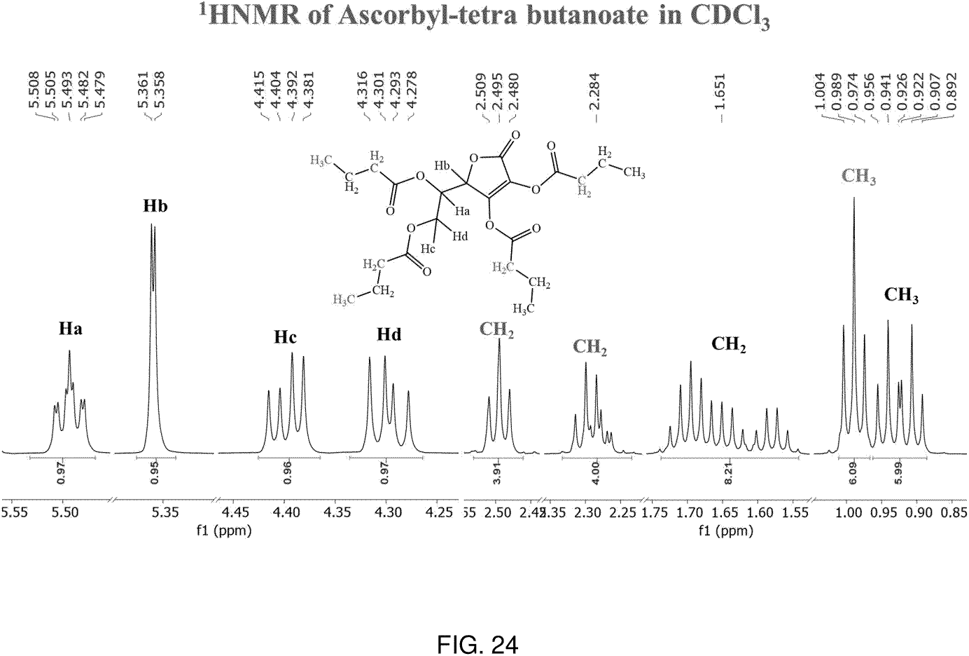

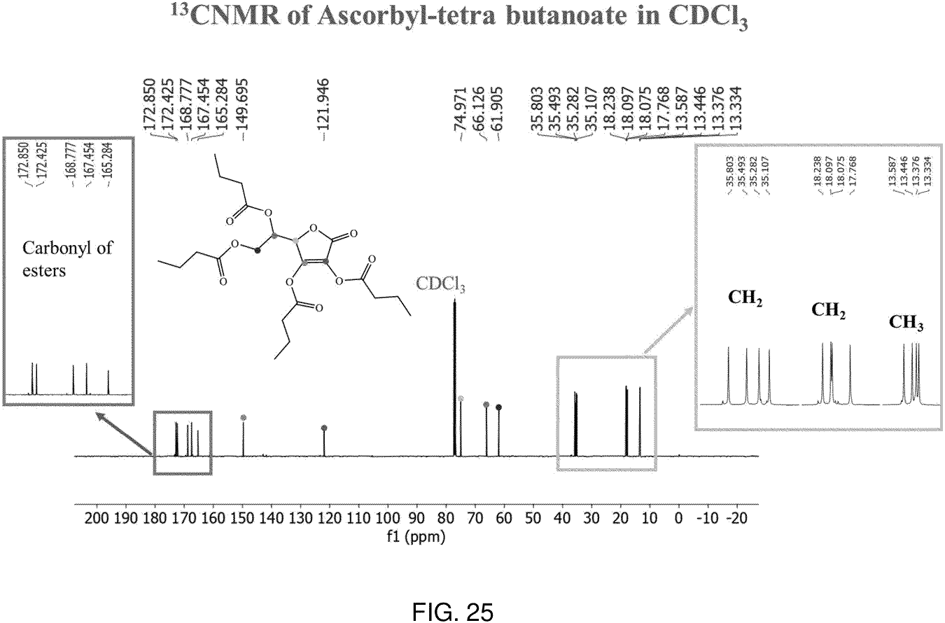

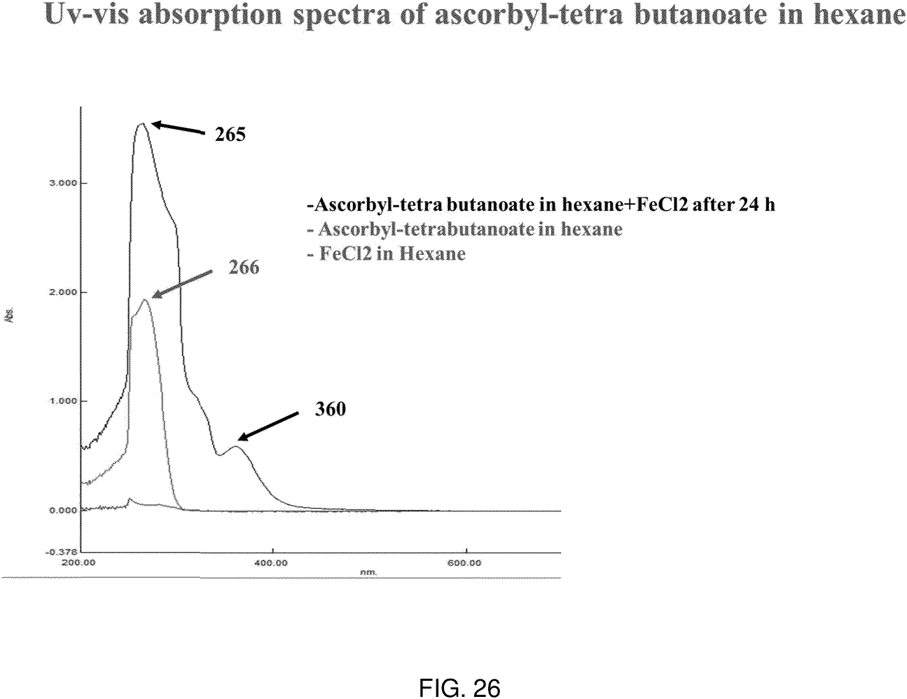

To a round bottom flask with a magnetic stir bar, septa, and nitrogen inlet, 200 mg (1.14 mmol) of catechin, 2.23 mL (13.6 mmol) of butyric anhydride, 35 mg (0.28 mmol) of 4-dimethylamino pyridine, and 6 mL of acetonitrile were added. The reaction mixture was stirred at room temperature for 24 h under a nitrogen blanket. The progress of the reaction was followed by thin-layer chromatography (TLC). After completing the reaction, the excess amount of butyric anhydride was evaporated and the crude product was extracted with 30 mL of ethyl acetate. Then, the organic phase was washed with (4 × 20 mL) of HCl (2 M). After that, the organic phase was washed with (3 × 20 mL) of NaHCO₃ (0.4 M). Finally, the organic solvent was dried with sodium sulfate and evaporated by a rotary evaporator to obtain the product as a light-orange viscose liquid in 64% yield (333 mg). FIGS. 22-26 show preparation and characterization of ascorbyl esters.

While there have been shown and described what are at present considered the preferred embodiments of the invention, those skilled in the art may make various changes and modifications which remain within the scope of the invention defined by the appended claims and the examples below.

1. A composition comprising:
an edible oil;
ionic iron; and
a curcumin ester.

2. The composition of claim 1, wherein the edible oil comprises coconut oil, corn oil, canola oil, palm oil, grape seed oil, soybean, peanut oil, olive oil, and combinations thereof.

3. The composition of claim 1, wherein the ionic iron comprises Fe²⁺.

4. The composition of claim 1, wherein the ionic iron is provided by addition of one or more of FeCl₂•4H₂O, FeSO4: FeSO₄: ferrous sulfate; iron(II) sulfate FeCl₂: ferrous chloride; iron(II) chloride Fe(NO₃)₃: ferric nitrate; iron(III) nitrate Fe(SO₄)₃: ferric sulfate; iron(III) sulfate FeCl₃: ferric chloride; iron(III) chloride.

5. The composition of claim 1, wherein the curcumin ester comprises curcumin diacetate, curcumin dipropanoate, curcumin dibutanoate, curcumin dipentanoate, curcumin dihexanoate, curcumin dioctanoate, curcumin didecanoate, curcumin didecanoate, curcumin ditetradecanoate, curcumin dihexadecanoate, curcumin dioctadecanoate, and combinations thereof.

6. The composition of claim 1, wherein the curcumin ester comprises curcumin monoacetate, curcumin monopropanoate, curcumin monobutanoate, curcumin monopentanoate, curcumin monohexanoate, curcumin monooctanoate, curcumin monodecanoate, curcumin monodecanoate, curcumin monotetradecanoate, curcumin monohexadecanoate, curcumin monooctadecanoate, and combinations thereof.

7. The composition of claim 1, wherein the curcumin ester and the ionic iron comprise a molar ratio of 1000:1 to 1:1 in the composition.

8. The composition of claim 1, wherein the curcumin ester comprises a concentration of at least 0.01 mg/ml in the composition.

9. The composition of claim 1, wherein the curcumin ester comprises a concentration of at least 0.01 wt% in the composition.

10. The composition of claim 1, wherein the iron comprises a concentration of at least 0.001 mg/ml in the composition.

11. The composition of claim 1, wherein the iron comprises a concentration of at least 0.001 wt% in the composition.

12. The composition of claim 1, further comprising a cosolvent.

13. The composition of claim 12, wherein the cosolvent is an alcohol.

14. The composition of claim 12, wherein the cosolvent is ethanol.

15. The composition of claim 13, wherein the cosolvent is at a concentration of 0.1 - 90% wt%.

16. The composition of claim 13, wherein the cosolvent is at a concentration of 2% wt%.

17-27. (canceled)

28. A method of preparing an iron-oil complex comprising:
combining curcumin ester and edible oil to form a curcumin ester and an edible oil mixture;
dispersing ionic iron in a cosolvent to form a dispersed ionic iron mixture; and
adding the dispersed ionic iron mixture to the curcumin ester and oil mixture.

29. The method of claim 28, wherein the edible oil coconut oil, corn oil, canola oil, palm oil, grape seed oil, soybean, peanut oil, olive oil, and combinations thereof.

30. The method of claim 28, wherein the ionic iron comprises Fe²⁺.

31. The method of claim 28, wherein the ionic iron is provided by addition of one or more of FeCl_{2•}4H₂O, FeSO4: FeSO₄: ferrous sulfate; iron(II) sulfate FeCl₂: ferrous chloride; iron(II) chloride Fe(NO₃)₃: ferric nitrate; iron(III) nitrate Fe(SO₄)₃: ferric sulfate; iron(III) sulfate FeCl₃: ferric chloride; iron(III) chloride.

32. The method of claim 28, wherein the curcumin ester comprises curcumin diacetate, curcumin dipropanoate, curcumin dibutanoate, curcumin dipentanoate, curcumin dihexanoate, curcumin dioctanoate, curcumin didecanoate, curcumin didecanoate, curcumin ditetradecanoate, curcumin dihexadecanoate, curcumin dioctadecanoate, and combinations thereof.

33. The method of claim 28, wherein the curcumin ester comprises curcumin monoacetate, curcumin monopropanoate, curcumin monobutanoate, curcumin monopentanoate, curcumin monohexanoate, curcumin monooctanoate, curcumin monodecanoate, curcumin monodecanoate, curcumin monotetradecanoate, curcumin monohexadecanoate, curcumin monooctadecanoate, and combinations thereof.

34. The method of claim 28, wherein the curcumin ester and the ionic iron comprise a molar ratio of 1000:1 to 1:1 in the composition.

35. The method of claim 28, wherein the curcumin ester comprises a concentration of at least 0.01 mg/ml in the composition.

36. The method of claim 28, wherein the curcumin ester comprises a concentration of at least 0.01 wt% in the composition.

37. The method of claim 28, wherein the iron comprises a concentration of at least 0.001 mg/ml in the composition.

38. The method of claim 28, wherein the iron comprises a concentration of at least 0.001 wt% in the composition.

39. The method of claim 28, wherein the cosolvent is an alcohol.

40. The method of claim 28, wherein the cosolvent is ethanol.

41. The method of claim 28, wherein the cosolvent is at a final concentration of 0.1 - 90% wt% in the iron-oil complex.

42. The method of claim 28, wherein the cosolvent is at a final concentration of 2% wt% in the iron-oil complex.

43-45. (canceled)

46. A method of treating an iron deficiency in a subject, the method comprising administering a therapeutic amount of the composition of claim 1.

47-138. (canceled)

139. A composition comprising:
an edible oil;
ionic iron; and
an esterified natural product.

140. The composition of claim 139, wherein the esterified natural product contains at least two groups capable of forming complexing coordination bonds with the ionic iron.

141. The composition of claim 140, wherein the esterified natural product is selected from the group consisting of esterified curcumin, catechin, and ascorbic acid.
